(19) United States
(12) Patent Application Publication
(10) Pub. No.: US 2024/0358643 A1
(43) Pub. Date: Oct. 31, 2024

(54) AN AMORPHOUS SOLID DISPERSION OF A QUINOLONE COMPOUND AND PROCESS FOR THE PREPARATION THEREOF

(71) Applicant: ZYDUS LIFESCIENCES LIMITED, Ahmedabad (IN)

(72) Inventors: Kumar Kamlesh SINGH, Ahmedabad (IN); Santosh Devidas DIWAKAR, Ahmedabad (IN); Sumer Singh CHUNDAWAT, Ahmedabad (IN); Jayesh Anilkumar SHARMA, Ahmedabad (IN); Ankit Nanjibhai JALELA, Ahmedabad (IN); Chintan DHOLAKIA, Ahmedabad (IN)

(21) Appl. No.: 18/292,235

(22) PCT Filed: Aug. 26, 2022
(86) PCT No.: PCT/IB2022/057993

§ 371 Date: Jan. 25, 2024

(30) Foreign Application Priority Data
Aug. 27, 2021 (IN) 202121038872

(51) Int. Cl. A61K 9/16; A61K 31/47

(52) U.S. Cl. CPC A61K 9/1652; A61K 9/1635; A61K 31/47

(57) ABSTRACT

The invention relates to an amorphous solid dispersion of a quinolone compound of Formula I and process for the preparation thereof.

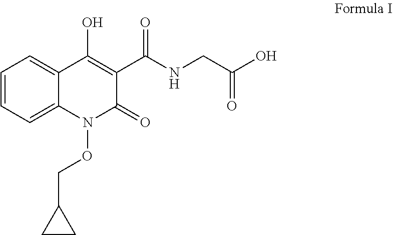 [SMILES: O=C(O)CNC(=O)c1c(O)c2ccccc2n(OCC2CC2)c1=O]

### RELATED APPLICATION

This application claims priority to Indian Provisional Patent Application No. 202121038872, filed on Aug. 27, 2021, the contents of which are incorporated herein in its entirety by reference.

### FIELD OF THE INVENTION

The invention relates to the field of pharmaceuticals, and in particular to an amorphous solid dispersion of a quinolone compound and process for the preparation thereof.

### BACKGROUND OF THE INVENTION

The following discussion of background is intended to present the invention in an appropriate technical context and allow its significance to be properly appreciated.

International (PCT) Publication No. WO 2014/102818 A1 discloses quinolone derivatives, including (1-(cyclopropylmethoxy)-4-hydroxy-2-oxo-1,2-dihydroquinoline-3-carbonyl)glycine, a compound of Formula (I),

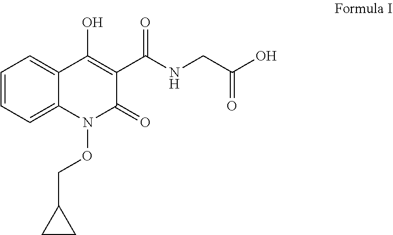 [SMILES: O=C(O)CNC(=O)c1c(O)c2ccccc2n(OCC2CC2)c1=O]

as Hypoxia-inducible factor (HIF) hydroxylases inhibitors having potential utility in any disease state where ischemia hypoxia and/or anemia plays a role.

U.S. PG-Pub. No. 2019/0359574 A1 discloses process for preparation of quinolone compounds including the compound of Formula I, and a crystalline form thereof.

The solid form of a compound plays a pivotal role in the formulation of pharmaceutical compositions. For example, different forms of a compound can have different physical properties (e.g., stability, dissolution rate, density, etc.) relating to their suitability for use in pharmaceutical compositions. Different polymorphic forms can also show different behavior with respect to their dissolution properties, flow properties, particle size distribution and chemical stability. Thus, having a suitable polymorphic form with desired properties is an important prerequisite during drug development.

An amorphous form generally provides better solubility and bioavailability than the crystalline form and may be useful for formulations. However pure amorphous drug forms tend to be unstable. As amorphous forms are thermodynamically unstable relative to the corresponding crystal forms, the amorphous forms would revert back to the stable crystalline form. This usually occurs during storage under various humidity and temperature conditions.

Therefore, it is desirable to have a stable amorphous form of drug with high purity to meet the needs of regulatory agencies and highly reproducible processes for its preparation.

The present invention provides the compound of Formula I in an amorphous solid dispersion form to aid in development of pharmaceutical composition and a process for the preparation thereof.

### SUMMARY OF THE INVENTION

In one general aspect, the present invention provides an amorphous solid dispersion comprising a compound of Formula I.

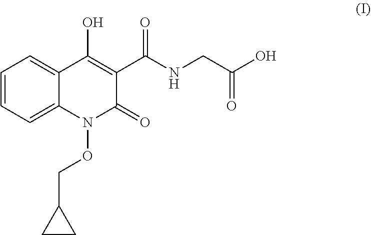 [SMILES: O=C(O)CNC(=O)c1c(O)c2ccccc2n(OCC2CC2)c1=O]

In another general aspect, the present invention provides an amorphous solid dispersion comprising a compound of Formula I, and

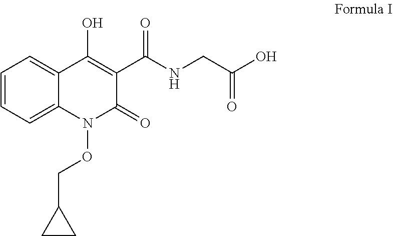 [SMILES: O=C(O)CNC(=O)c1c(O)c2ccccc2n(OCC2CC2)c1=O]

one or more pharmaceutically acceptable carriers.

In another general aspect, the present invention provides a process for the preparation of an amorphous solid dispersion comprising a compound of Formula I and one or more pharmaceutically acceptable carriers, the process comprising:
(a) preparing a solution of the compound of Formula I together with one or more pharmaceutically acceptable carriers in one or more solvents; and
(b) obtaining the amorphous solid dispersion by the removal of solvent.

In another general aspect, the present invention provides a pharmaceutical composition comprising an amorphous solid dispersion of a compound of Formula I and one or more pharmaceutically acceptable excipients.

The amorphous solid dispersion of the compound of Formula I was found to be stable upon storage and exhibits increased solubility. The amorphous solid dispersion of compound of Formula I of the present invention may provide better oral bioavailability and/or a better dissolution profile for a particular formulation; may also provide free-flowing, easily filterable, and/or thermally stable characteristics that are suitable for use in particular formulations.

### BRIEF DESCRIPTION OF THE FIGURES AND DRAWINGS

FIG. 1. X-Ray powder diffraction (XRPD) pattern of an amorphous solid dispersion of compound of Formula I together with HPMC prepared according to example 1.

FIG. 2. X-Ray powder diffraction (XRPD) pattern of an amorphous solid dispersion of compound of Formula I together with HPMC prepared according to example 2.

FIG. 3. X-Ray powder diffraction (XRPD) pattern of an amorphous solid dispersion of compound of Formula I together with HPMC prepared according to example 3.

FIG. 4. X-Ray powder diffraction (XRPD) pattern of an amorphous solid dispersion of compound of Formula I together with co-povidone prepared according to example 4.

FIG. 5. X-Ray powder diffraction (XRPD) pattern of an amorphous solid dispersion of compound of Formula I together with co-povidone prepared according to example 5.

FIG. 6. X-Ray powder diffraction (XRPD) pattern of an amorphous solid dispersion of compound of Formula I together with co-povidone prepared according to example 6.

FIG. 7. X-Ray powder diffraction (XRPD) pattern of an amorphous solid dispersion of compound of Formula I together with HPMC-AS prepared according to example 7.

FIG. 8. X-Ray powder diffraction (XRPD) pattern of an amorphous solid dispersion of compound of Formula I together with Kollidon® SR prepared according to example 8.

FIG. 9. X-Ray powder diffraction (XRPD) pattern of an amorphous solid dispersion of compound of Formula I together with Kollidon® SR prepared according to example 9.

FIG. 10. X-Ray powder diffraction (XRPD) pattern of an amorphous solid dispersion of compound of Formula I together with Eudragit® RS-PO prepared according to example 10.

### DETAILED DESCRIPTION OF THE INVENTION

The above and other objects of the present invention are achieved by the process of the present invention, which leads a process for the preparation of an amorphous solid dispersion of compound of Formula I.

Optionally, the solution, prior to any solids formation, can be filtered to remove any undissolved solids or solid impurities prior to removal of the solvent. Any filtration system and filtration techniques known in the art can be used.

All ranges recited herein include the endpoints, including those that recite a range “between” two values.

The term “substantially” is to be construed as modifying a term or value such that it is not an absolute. This includes, at very least, the degree of expected experimental error, technique error and instrument error for a given technique used to measure a value.

The term “solid dispersion” as used herein, means a molecular dispersion of a compound, particularly a drug substance within a carrier. The term solid dispersion in general means a system in solid state comprising at least two components, wherein one component is dispersed substantially evenly throughout the other component(s). In certain embodiments, a solid dispersion as disclosed herein includes an active ingredient compound of Formula I dispersed among at least one other component-a pharmaceutically acceptable carrier, for example a polymer.

The terms “carrier” and “pharmaceutically acceptable carrier” as used herein are interchangeable. The carrier is able to form a matrix embedding (surrounding) the active ingredient. The matrix may comprise one carrier or a mixture of two or more carriers. The carrier used in the solid dispersion of the present invention may be a polymer.

The term “pharmaceutical composition” as used herein, is intended to encompass a drug product including the active ingredient(s), pharmaceutically acceptable excipients that make up the carrier, as well as any product, which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The particle size is determined on a particle volume basis by using techniques known for the measurement of particle size. Particle size can be characterized by one or more values such as D₉₀, D₅₀ or D₁₀.

The term “D₉₀” as used herein, describes the value of particle size at which 90% of the total volume of particles is comprised of particles of the indicated size.

The term “D₅₀” as used herein, describes the value of particle size at which 50% of the total volume of particles is comprised of particles of the indicated size.

The term “D₁₀” as used herein, describes the value of particle size at which 10% of the total volume of particles is comprised of particles of the indicated size.

The term “about” as used herein, is intended to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “pharmaceutically acceptable” as used herein means useful in preparing a pharmaceutical composition that is generally non-toxic and is not biologically undesirable, and is acceptable for veterinary or human pharmaceutical use.

The term “composition” as used herein means a physical mixture of two or more components.

The term “method of treatment” as used herein, means any treatment of a disease or disorder in a mammal, including preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms.

The term “stable” as used herein refers to the polymorphic form stability and chemical stability.

The term “amorphous solid dispersion of compound of Formula I” as used herein means the amorphous solid dispersion comprising compound of Formula I and one or more pharmaceutically acceptable carriers.

In general, the compound of Formula I to be used as the starting material may be prepared by the known methods reported in the prior art, for example, by using the process as disclosed in U.S. PG-Pub. No. 2019/0359574 A1, which is incorporated herein as reference.

Thus, in one general aspect, the present invention provides an amorphous solid dispersion comprising a compound of Formula I, and

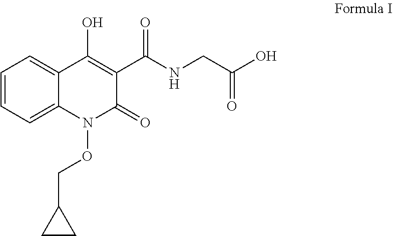 [SMILES: O=C(O)CNC(=O)c1c(O)c2ccccc2n(OCC2CC2)c1=O]

one or more pharmaceutically acceptable carriers.

In general, the pharmaceutically acceptable carrier is a polymer. The polymer may be a non-ionic polymer or an ionic polymer. In an embodiment, the one or more pharmaceutically acceptable carrier in the amorphous solid dispersion of the present invention is polymer selected from hydroxypropyl methylcellulose (HMPC), polyvinylpyrrolidone (PVP) or PVP based polymers (such as Kollidon® SR, Kollidon® 90), co-povidone, hydroxypropyl methylcellulose acetate succinate (HPMC-AS), hydroxypropyl cellulose, ethyl cellulose, carboxymethyl cellulose, polyethylene glycol, and copolymers based on methacryalic acid and methacrylic/acrylic ester or derivatives (such as Eudragit® RS-PO), or a mixture thereof.

In particular, PVP of different grades like K-15, K-30, K-60, K-90 and K-120 may be used for the preparation of amorphous dispersion. PVP based polymers such as Kollidon® SR and Kollidon® 90 may be used. Particularly, the copolymers based on methacryalic acid and methacrylic/acrylic ester or derivatives can be selected from the polymers manufactured by Evonik under the trade name Eudragit® such as Eudragit® RS-PO. More particularly, hydroxypropylmethyl cellulose (HPMC) or its acetate succinate and PVP K-30 may be used. HPMC with viscosity 8 cps, 5 cps or 3 cps may be used. In particular, the pharmaceutically acceptable carrier is HPMC or co-povidone.

In another embodiment, the one or more pharmaceutically acceptable carrier in the amorphous solid dispersion of the present invention is selected from hydroxypropyl methylcellulose, co-povidone, hydroxypropyl methylcellulose acetate succinate (HPMC-AS), polyvinylpyrrolidone (PVP) or PVP based polymers (such as Kollidon® SR, Kollidon® 90) and copolymers based on methacrylic acid and methacrylic/acrylic ester or derivatives (such as Eudragit® RS-PO).

In another embodiment, the pharmaceutically acceptable carrier is HPMC with viscosity 3cps.

In another general aspect, the present invention provides an amorphous solid dispersion comprising a compound of Formula I and one or more pharmaceutically acceptable carriers, wherein the ratio of compound of Formula I to the pharmaceutically acceptable carrier is about 1:1 to 1:10 w/w. Particularly, the ratio of the compound of Formula I to the pharmaceutically acceptable carrier is about 1:1 to about 1:5 w/w. More particularly, the ratio of the compound of Formula I to the pharmaceutically acceptable carrier is about 1:1 to about 1:3 w/w.

In another general aspect, the present invention provides an amorphous solid dispersion comprising a compound of Formula I and one or more pharmaceutically acceptable carriers, wherein the pharmaceutically acceptable carrier is selected from hydroxypropyl methylcellulose (HPMC), co-povidone, hydroxypropyl methylcellulose acetate succinate (HPMC-AS), Kollidon® SR, Kollidon® 90 and Eudragit® RS-PO and wherein the ratio of compound of Formula I to the pharmaceutically acceptable carrier is about 1:1 w/w to 1:3 w/w.

In another general aspect, the present invention provides an amorphous solid dispersion comprising a compound of Formula I and a pharmaceutically acceptable carrier, wherein the pharmaceutically acceptable carrier is HPMC and the ratio of compound of Formula I to the pharmaceutically acceptable carrier is about 1:3 w/w.

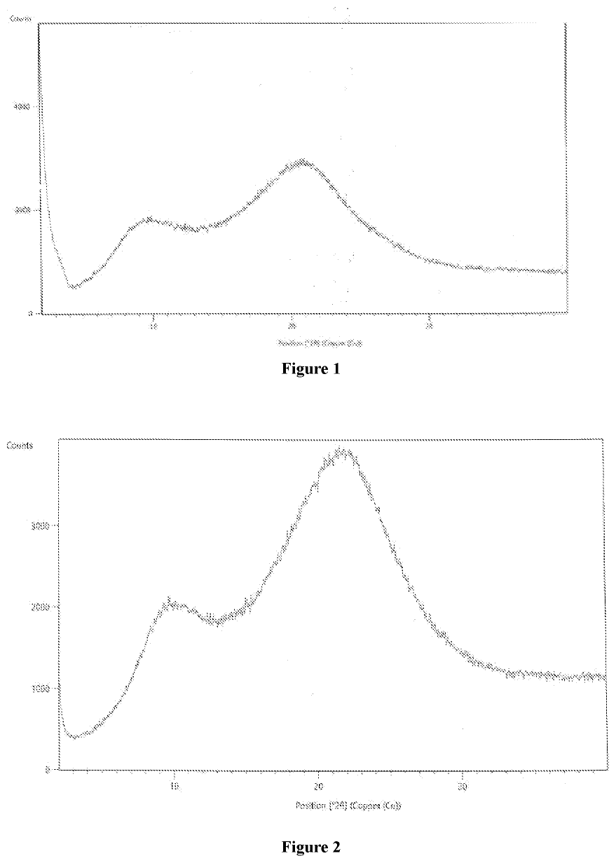

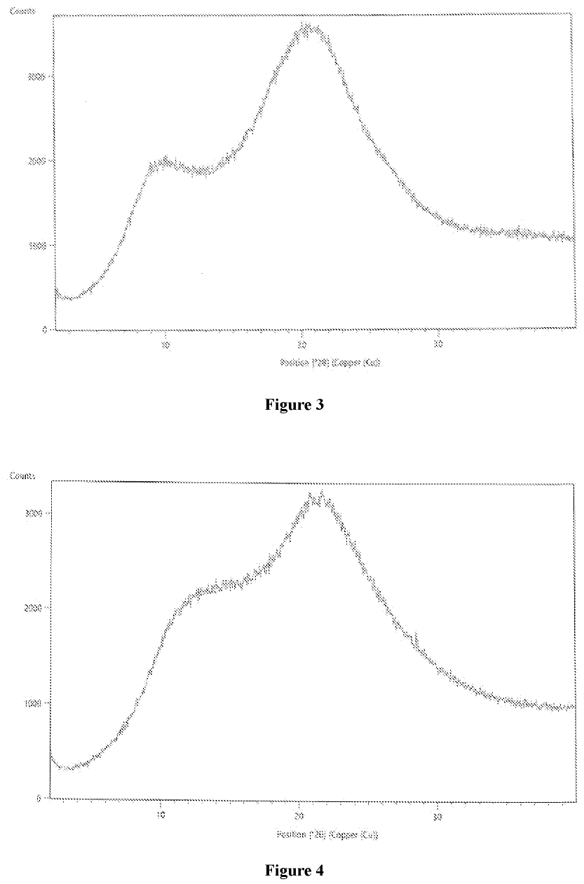

In another general aspect, the present invention provides an amorphous solid dispersion of the compound of Formula I together with HPMC characterized by an X-Ray powder diffraction (XRPD) pattern substantially as same as depicted in FIG. 1, FIG. 2 or FIG. 3.

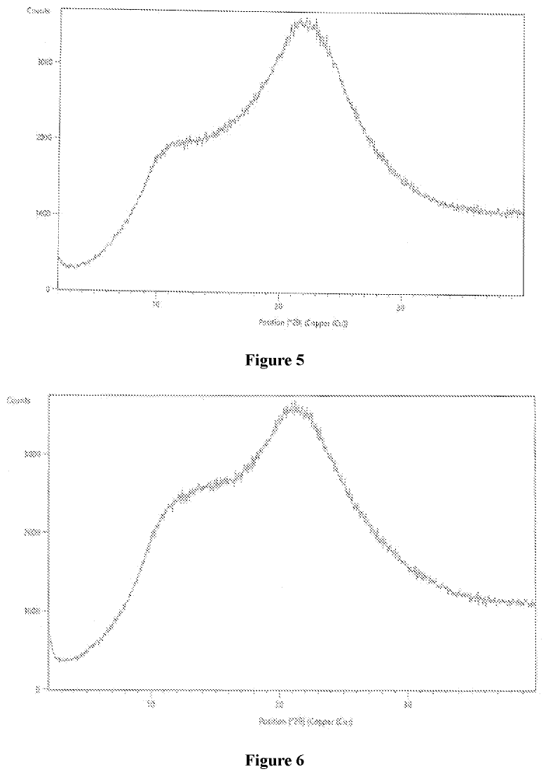

In another general aspect, the present invention provides an amorphous solid dispersion of the compound of Formula I together with co-povidone characterized by an X-Ray powder diffraction (XRPD) pattern substantially as same as depicted in FIG. 4, FIG. 5 or FIG. 6.

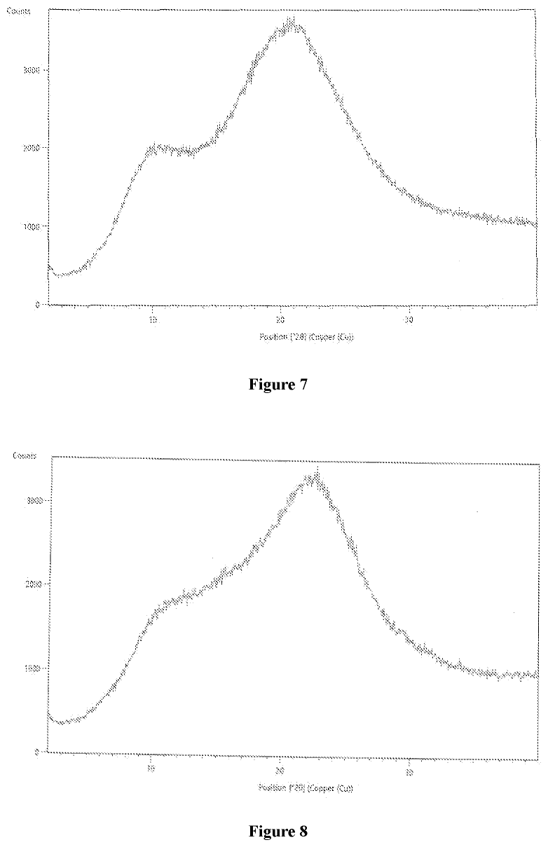

In another general aspect, the present invention provides an amorphous solid dispersion of the compound of Formula I with HPMC-AS characterized by an X-Ray powder diffraction (XRPD) pattern substantially as same as depicted in FIG. 7.

In another general aspect, the present invention provides an amorphous solid dispersion of the compound of Formula I with Kollidon® SR characterized by an X-Ray powder diffraction (XRPD) pattern substantially as same as depicted in FIG. 8.

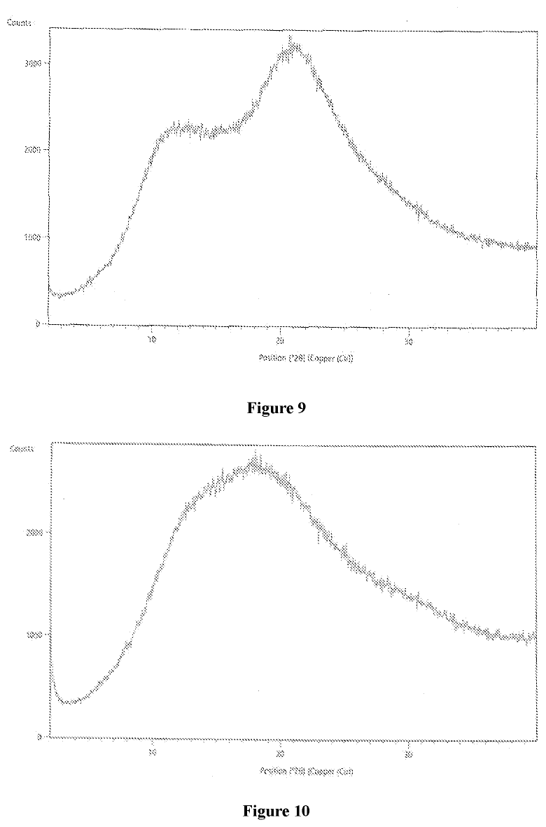

In another general aspect, the present invention provides an amorphous solid dispersion of the compound of Formula I with Kollidon® 90 characterized by an X-Ray powder diffraction (XRPD) pattern substantially as same as depicted in FIG. 9.

In another general aspect, the present invention provides an amorphous solid dispersion of the compound of Formula I with Eudragit® RS-PO characterized by an X-Ray powder diffraction (XRPD) pattern substantially as same as depicted in FIG. 10.

In another general aspect, the present invention provides an amorphous solid dispersion comprising a compound of Formula I and pharmaceutically acceptable carrier, wherein the pharmaceutically acceptable carrier is co-povidone and the ratio of compound of Formula I to the pharmaceutically acceptable carrier is about 1:3 w/w.

In another general aspect, the present invention provides an amorphous solid dispersion comprising a compound of Formula I and one or more pharmaceutically acceptable carriers, wherein the compound of Formula I is having a purity of about 98% or more, by area percentage of high-performance liquid chromatography (HPLC).

In another general aspect, the compound of Formula I in the amorphous solid dispersion is having a purity of about 99% or more, by area percentage of HPLC.

In another general aspect, the compound of Formula I in the amorphous solid dispersion is having a purity of about 99.5% or more, by area percentage of HPLC.

In another general aspect, the compound of Formula I in the amorphous solid dispersion is having a purity of about 99.8% or more, by area percentage of HPLC.

In another general aspect, the compound of Formula I in the amorphous solid dispersion is having a purity of about 99.9% or more, by area percentage of HPLC.

The amorphous solid dispersion of the compound of Formula I together with the pharmaceutically acceptable carrier of the present invention is stable during storage. This property is important and advantageous for the desired use of compound of Formula I in pharmaceutical product formulations.

The amorphous solid dispersion of the compound of Formula I together with pharmaceutically acceptable carrier of the present invention is stable when stored at 25±2° C./60±5% relative humidity (RH) and at 40±2° C./75±5% RH for a period of 3 months or more. The stability is measured by an absence of any crystallinity when the amorphous solid dispersion stored at 25±2° C./60±5% RH and at 40±2° C./75±5% RH for a period of 3 months or more.

Particularly, the amorphous solid dispersion of the compound of Formula I together with pharmaceutically acceptable carrier of the present invention is stable when stored at 2-8° C. or at 25±2° C./60±5% RH for a period of 15 days or more, wherein the amorphous solid dispersion does not show any crystallinity after storage.

Thus, in another general aspect, the present invention provides a stable amorphous solid dispersion of the compound of Formula I together with one or more pharmaceutically acceptable carriers.

In another general aspect, the present invention provides a process for the preparation of an amorphous solid dispersion comprising a compound of Formula I, and one or more pharmaceutically acceptable carriers, the process comprising:
(a) preparing a solution of the compound of Formula I together with one or more pharmaceutically acceptable carriers in one or more solvents; and
(b) obtaining the amorphous solid dispersion by the removal of solvent.

In general, at step a) of the above process, any physical form of the compound of Formula I may be utilized for providing the solution of compound of Formula I together with one or more pharmaceutically acceptable carrier in one or more organic solvents. The solution may be prepared by dissolving the compound of Formula I together with one or more pharmaceutically acceptable carrier in a suitable solvent or a mixture of solvent at a suitable temperature ranging from 0° C. to the reflux temperature of the solvent. In particular, the dissolution may be performed at a temperature of about 25° C. to about 120° C., more particularly, at a temperature of about 25° C. to about 35° C. so as to obtain the clear solution of compound of Formula I and one or more pharmaceutically acceptable carrier. In order to form a solution of step a), the contents may be stirred for sufficient period of time at a suitable temperature of about 25° C. to about reflux temperature of the solvent. Typically, the contents were stirred for about 10 min to about 2 hrs at a temperature of about 25° C. to about 35° C.

The one or more pharmaceutically acceptable carrier as per step (a) of the above process, comprises one or more of hydroxypropyl methylcellulose (HMPC), polyvinylpyrrolidone (PVP) or PVP based polymers (such as Kollidon® SR, Kollidon® 90), co-povidone, hydroxypropyl methylcellulose acetate succinate (HPMC-AS), hydroxypropyl cellulose, ethyl cellulose, carboxymethyl cellulose, polyethylene glycol, and copolymers based on methacrylic acid and methacrylic/acrylic ester or derivatives (such as Eudragit® RS-PO), or a mixture thereof. In particular, the one or more pharmaceutically acceptable carrier is selected from hydroxypropyl methylcellulose, co-povidone, hydroxypropyl methylcellulose acetate succinate (HPMC-AS), polyvinylpyrrolidone (PVP) or PVP based polymers (such as Kollidon® SR, Kollidon® 90) and copolymers based on methacrylic acid and methacrylic/acrylic ester or derivatives (such as Eudragit® RS-PO).

In an embodiment, the pharmaceutically acceptable carrier is HPMC. In another embodiment, the pharmaceutically acceptable carrier is co-povidone.

In general, the one or more solvent that may be used at step (a) of the above process comprises one or more of chlorinated hydrocarbon solvents such as dichloromethane, dichloroethane and chlorobenzene; alcoholic solvent such as methanol, ethanol, 2-propanol, 1-butanol, and t-butyl alcohol; N,N-dimethylformamide; dimethyl sulfoxide; water, and mixtures thereof.

In another general aspect, the solvent at step (a), is a mixture of a chlorinated hydrocarbon solvent and an alcoholic solvent. In general, the solvent is a mixture of dichloromethane and methanol. Particularly, the solvent is a mixture of dichloromethane and methanol, wherein the ratio of dichloromethane to methanol is 1:10 v/v to 10:1 v/v. More particularly, the ratio of dichloromethane to methanol is 1:1 v/v.

In general, the step (b) of the above process involves removal of the solvent to obtain an amorphous solid dispersion of the compound of Formula I. Techniques which may be used for the removal of solvent include one or more of distillation, distillation under vacuum, spray drying, agitated thin film drying (ATFD), freeze drying (lyophilization), filtration, decantation, and centrifugation.

The solvent may be removed, optionally under reduced pressures, at temperatures less than 70° C., less than 60° C., particularly less than 50° C.

In general, freeze drying (lyophilization) may be performed by freezing a solution of the compound of Formula I and one or more pharmaceutically acceptable carrier, at a low temperature and reducing the pressure to remove the solvent from the frozen solution of compound of Formula I and pharmaceutically acceptable carrier. Temperature that may be required to freeze the solution is depending on the solvent chosen to make the solution.

In particular, the removal of solvent as per step (b), can be performed by spray drying using a spray dryer. In general, spray drying involve spray drying of feed stock, which is prepared as discussed below. In particular, the spray drying of the solution of compound of Formula I and the pharmaceutically acceptable carrier, may be performed maintaining the inlet temperature in the range of 60° C. to 80° C., atomization at about 0.85 to 0.95 kg/cm², flow rate of about 8 to 10 mL/min, maintaining the outlet temperature in the range of 50° C. to 70° C., aspiration flow rate of about 60 to 75 Nm³/Hrs and maintaining the vacuum at −10 to 0 mm of WC.

In general, the feed stock of the compound of Formula I is conveniently prepared by dissolving any known solid forms or wet cake of compound of Formula I together with one or more pharmaceutically acceptable carriers, in one or more solvents selected from chlorinated hydrocarbon solvent such as dichloromethane, dichloroethane and chlorobenzene; alcoholic solvents such as methanol, ethanol, 2-propanol, 1-butanol, and t-butyl alcohol; N,N-dimethylformamide; dimethyl sulfoxide, and mixtures thereof. Particularly, the solvent for the purpose is a mixture of a chlorinated hydrocarbon solvent and an alcoholic solvent. More particularly, the solvent for the purpose is a mixture of dichloromethane and methanol. More particularly, the solvent is a mixture of dichloromethane and methanol, wherein the ratio of dichloromethane to methanol is 1:10 v/v to 10:1 v/v. More particularly, the ratio of dichloromethane to methanol is 1:1 v/v.

In another general aspect, the present invention provides a process for the preparation of amorphous solid dispersion of the compound of Formula I, the process comprising:
(a) preparing a solution of the compound of Formula I together with one or more pharmaceutically acceptable carriers in one or more solvents; and
(b) spray drying the said solution, thereby producing said amorphous solid dispersion.

The one or more pharmaceutically acceptable carriers and the solvents for the purpose are as described supra.

In another general aspect, the present invention provides a process for the preparation of amorphous solid dispersion of the compound of Formula I, the process comprising:
(a) preparing a solution of the compound of Formula I together with one or more pharmaceutically acceptable carriers in a mixture of chlorinated hydrocarbon solvents and alcoholic solvents; and
(b) spray drying the said solution, thereby producing said amorphous solid dispersion.

In yet another general aspect, the present invention provides a process for the preparation of amorphous solid dispersion of the compound of Formula I, the process comprising:
(a) preparing a solution of the compound of Formula I together with one or more pharmaceutically acceptable carriers in a mixture of dichloromethane and methanol; and
(b) spray drying the said solution, thereby producing said amorphous solid dispersion.

In another general aspect, the present invention provides an amorphous solid dispersion comprising a compound of Formula I, and one or more pharmaceutically acceptable carriers, wherein the compound of Formula I is having a particle size of D₉₀ less than about 250 μm.

In another general aspect, the present invention provides an amorphous solid dispersion comprising a compound of Formula I, and one or more pharmaceutically acceptable carriers, wherein the compound of Formula I is having a particle size of.
D₉₀ less than about 175 μm,
D₅₀ less than about 75 μm, and
D₁₀ less than about 10 μm.

In one embodiment, the compound of Formula I in the amorphous solid dispersion of the present invention is having a particle size of D₉₀ less than about 250 μm and D₅₀ greater than about 5 μm.

In another embodiment, the compound of Formula I in the amorphous solid dispersion of the present invention is having a particle size of D₉₀ less than about 200 μm.

In another embodiment, the compound of Formula I in the amorphous solid dispersion of the present invention is having a particle size of D₉₀ less than about 175 μm.

In another embodiment, the compound of Formula I in the amorphous solid dispersion of the present invention is having a particle size of D₉₀ less than about 100 μm.

In another embodiment, the compound of Formula I in the amorphous solid dispersion of the present invention is having a particle size of D₉₀ less than about 200 μm, for example, less than about 175 μm, less than about 150 μm, less than about 130 μm, less than about 110 μm, less than about 100 μm, less than 75 μm, less than 50 μm, or less than 30 μm.

In particular, the compound of Formula I in the amorphous solid dispersion of the present invention is having a particle size of D₉₀ less than about 50 μm.

In another embodiment, the compound of Formula I in the amorphous solid dispersion of the present invention is having a particle size of D₅₀ less than about 150 μm, for example less than about 100 μm, less than about 50 μm, less than about 40 μm, less than about 30 μm, less than about 20 μm or less than about 10 μm.

In another embodiment, the compound of Formula I in the amorphous solid dispersion of the present invention is having a particle size of D₁₀ less than about 30 μm.

In another embodiment, the compound of Formula I in the amorphous solid dispersion of the present invention is having a particle size of D₁₀ less than about 10 μm.

In another general aspect, the present invention provides an amorphous solid dispersion comprising a compound of Formula I, and one or more pharmaceutically acceptable carrier, wherein the compound of Formula I is having a particle size of.
D₉₀ less than about 50 μm,
D₅₀ less than about 30 μm, and
D₁₀ less than about 10 μm.

In another general aspect, the present invention provides a composition comprising an amorphous solid dispersion of compound of Formula I having a purity of about 98% or more by area percentage of high-performance liquid chromatography (HPLC), together with one or more pharmaceutically acceptable carriers, and one or more of compounds of Formulae A, B, C, D or E present in an amount less than about 0.15% by area percentage of HPLC relative to the compound of Formula (I),

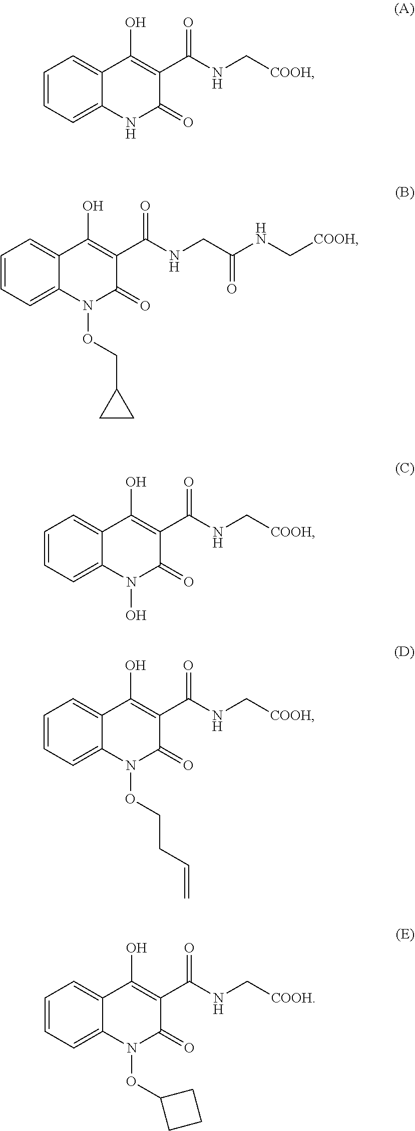 [SMILES: O=C(O)CNC(=O)c1c(O)c2ccccc2[nH]c1=O ; O=C(O)CNC(=O)CNC(=O)c1c(O)c2ccccc2n(OCC2CC2)c1=O ; O=C(O)CNC(=O)c1c(O)c2ccccc2n(O)c1=O ; C=CCCOn1c(=O)c(C(=O)NCC(=O)O)c(O)c2ccccc21 ; O=C(O)CNC(=O)c1c(O)c2ccccc2n(OC2CCC2)c1=O]

In general, the present invention provides a composition comprising an amorphous solid dispersion of compound of Formula I having a purity of about 98% or more, about 99% or more, about 99.5% or more, about 99.8% or more, or about 99.9% or more by area percentage of high-performance liquid chromatography (HPLC), together with one or more pharmaceutically acceptable carriers, and one or more of compounds of Formulae A, B, C, D or E present in an amount less than about 0.15% by area percentage of HPLC relative to the compound of Formula (I).

In particular, the impurity content for each of the impurities of compound of Formulae A, B, C, D, and E by area percentage of HPLC is about 0.15% or less, more particularly, about 0.10% or less, or more particularly not detected by HPLC method of analysis.

In another general aspect, the present invention provides a composition comprising an amorphous solid dispersion of compound of Formula I having a purity of about 99% or more by area percentage of high-performance liquid chromatography (HPLC), together with one or more pharmaceutically acceptable carriers, and one or more of compounds of Formulae C, D or E present in an amount less than about 0.15% by area percentage of HPLC relative to the compound of Formula (I).

In another general aspect, the present invention provides a composition comprising an amorphous solid dispersion of compound of Formula I having a purity of about 99% or more by area percentage of high-performance liquid chromatography (HPLC), together with one or more pharmaceutically acceptable carriers, and the compound of Formula D present in an amount less than about 0.15% by area percentage of HPLC relative to the compound of Formula (I).

In another general aspect, the present invention provides a composition comprising an amorphous solid dispersion of compound of Formula I having a purity of about 99% or more by area percentage of high-performance liquid chromatography (HPLC), together with one or more pharmaceutically acceptable carriers, and the compound of Formula E present in an amount less than about 0.15% by area percentage of HPLC relative to the compound of Formula (I).

In another aspect, the amorphous solid dispersion of compound of Formula I obtained is formulated into a finished dosage form.

In general, the finished dosage form comprises one or more of liquid, solid and semisolid dosage forms depending upon the route of administration.

Thus, in another general aspect, the present invention provides a pharmaceutical composition comprising an amorphous solid dispersion of compound of Formula I and one or more pharmaceutically acceptable excipients.

In another general aspect, the present invention provides a pharmaceutical composition comprising an amorphous solid dispersion of compound of Formula I and one or more pharmaceutically acceptable excipients, wherein the compound of Formula I is having a purity of about 98% or more, by area percentage of high-performance liquid chromatography (HPLC).

In general, the present invention provides a pharmaceutical composition comprising an amorphous solid dispersion of compound of Formula I and one or more pharmaceutically acceptable excipients, wherein the compound of Formula I is having a purity of about 98% or more, about 99% or more, about 99.5% or more, about 99.8% or more, or about 99.9% or more by area percentage of HPLC.

In another general aspect, the present invention provides a method for the treatment of anemia in a patient, comprising administering to a patient in need thereof a pharmaceutical composition comprising an amorphous solid dispersion of the compound of Formula I and one or more pharmaceutically acceptable excipients.

The present invention is further illustrated by the following example which is provided merely to be exemplary of the invention and do not limit the scope of the invention. Certain modification and equivalents will be apparent to those skilled in the art and are intended to be included within the scope of the present invention. The examples are set forth to aid in understanding the invention but are not intended to, and should not be construed to limit its scope in any way. The examples do not include detailed descriptions of conventional methods. Such methods are well known to those of ordinary skill in the art and are described in various publications.

### EXAMPLES

### (i) Characterization by X-Ray Powder Diffraction

The X-ray powder diffraction spectrum was measured under the following experimental conditions:
Instrument: X-Ray Diffractometer, PW3050/60, Make: PANalytical.
   X-Ray: Cu K alpha radiation
   Tension: 45 KV
   Current: 40 mA
   Divergence slit: Automatic
Incident beam side
   Off set: 0.000
   Anti-scatter slit: ½°
   Receiving slit: None
   Detector: PIXcel1D-Medipix3
   Mode: Scanning line detector (1D)
Method parameter
   Start position: 2° 2θ
   End position: 40° 2θ
   Step size: 0.02° rad
   Time per step: 67.575s
   Scan mode: Continuous

### (ii) HPLC Analysis Method:

The HPLC purity of the compound of Formula (I) was calculated using following method: Instrument Name: Waters Alliance e2695 HPLC system or equivalent

### HPLC Chromatographic Conditions:

| Column: | Zorbax SB-Phenyl (150 mm × 4.6 mm, 5 μm) | |
|---|---|---|
| | Ghost hunter column (4.6 mm × 53 mm, Part | |
| | No.: 0646PRONTOGHO, Make: Prontosil) or | |
| | equivalent | |
| Detector | UV-VIS or PDA detector | |
| Flow rate | 2.0 | mL/min |
| Wavelength | 230 | nm |
| Injection volume | 10 | μL |
| Column temperature | 30° | C. |
| Run time | 70 | minutes |
| Sample cooler | 25° | C. |
| temperature | | |

| Time (minutes) | % Mobile phase A | % Mobile phase B |
|---|---|---|
| 0 | 82 | 18 |
| 18 | 82 | 18 |
| 40 | 40 | 60 |
| 50 | 30 | 70 |
| 60 | 30 | 70 |
| 63 | 82 | 18 |
| 70 | 82 | 18 |

Preparation of Dilute Orthophosphoric acid solution:
10 mL of Orthophosphoric acid was transferred into 100 mL volumetric flask and the volume was make up to the mark with Milli-Q water and mixed well.

Preparation of buffer:
1.36 g of potassium dihydrogen phosphate was transferred in to suitable container. About 1000 mL of Milli Q water was added and sonicated to dissolve the content and mixed well. The pH 3.5±0.05 was adjusted with controlled addition of dilute Orthophosphoric acid solution and filtered through 0.45 μm PVDF membrane filter paper.

Mobile phase-A:
Degassed mixture of Buffer: Methanol in the volume ratio of 95:05 (v/v).

Mobile phase-B:
Degassed mixture of Acetonitrile: Buffer in the volume ratio of 70:30 (v/v).

Diluent:
Degassed mixture of Acetonitrile: Methanol in the volume ratio of 50:50 (v/v)

Blank: diluent as a blank.

(iii) Spray Dryer Parameter:
Instrument Name: Labultima LU 222 Advance
Inlet Temperature: 70 to 75° C.
Outlet Temperature: 55 to 65° C.
Product Higher Temperature: 65° C.
Niddle Size: 0.7 mm
Atomization: 0.85 to 0.95 Kg/Cm²
Flow rate: 8 to 10 ml/min
Vacuum: −10 to 0 mm of WC
Aspiration Flow Rate: 60 to 75 Nm³/Hrs
Oxygen Level: 3 to 3.5%

### (iv) Particle Size Determination:

Particle size was determined by Malvern Mastersizer 3000 with Hydro MV accessory, laser diffraction particle size analyser. About 100 mg of the sample weighed and transferred into 100 mL of glass beaker. About 10-15 drops of dispersant sunflower oil: cyclohexane (80:20) were added and the lumps were broken with glass rod for about 3 to 5 min. 20 mL of dispersant was added into the same glass beaker and the content were shaken for 3-5 minutes with glass rod to mix well. Background measurement using dispersant was performed. When the blank correction/background measurement was over, sample slurry (with continuous manual shaking) was added in the sampler at 1500 rpm to get the target obscuration value between 10% and 30% and remained constant. Then the histogram was recorded.

### Example 1. Preparation of Amorphous Solid Dispersions of Compound of Formula I with HPMC (1:3 w/w)

In a round bottom flask, a mixture of (1-(cyclopropylmethoxy)-4-hydroxy-2-oxo-1,2-dihydroquinoline-3-carbonyl)glycine (5 g) and HPMC (3cps) (15 g) were dissolved in a mixture of methanol (100 mL) and dichloromethane (100 mL) at 25-35° C. The clear solution was fine filtered and used for spray drying. The solution was then spray dried to get an amorphous solid dispersion of (1-(cyclopropylmethoxy)-4-hydroxy-2-oxo-1,2-dihydroquinoline-3-carbonyl) glycine with HPMC.

The XRPD pattern is as set forth in FIG. 1.

### Example 2. Preparation of Amorphous Solid Dispersions of Compound of Formula I with HPMC (1:1 w/w)

The amorphous solid dispersions of compound of Formula I with HPMC (1:1 w/w) was prepared by following the similar process as described in example 1 above taking the ratio compound of Formula I with HPMC (1:1 w/w).

The XRPD pattern is as set forth in FIG. 2.

### Example 3. Preparation of Amorphous Solid Dispersions of Compound of Formula I with HPMC (1:3 w/w)

The amorphous solid dispersions of compound of Formula I with HPMC (6 cps) (1:3 w/w) was prepared by following the similar process as described in example 1.

The XRPD pattern is as set forth in FIG. 3.

### Example 4. Preparation of Amorphous Solid Dispersions of Compound of Formula I with Co-Povidone (1:3 w/w)

A mixture of (1-(cyclopropylmethoxy)-4-hydroxy-2-oxo-1,2-dihydroquinoline-3-carbonyl)glycine (5 g) and co-povidone (15 g) were dissolved in a mixture of methanol (200 mL) and dichloromethane (200 mL) at 25-35° C. The clear solution was fine filtered and used for spray drying. The solution was then spray dried to get an amorphous solid dispersion of (1-(cyclopropylmethoxy)-4-hydroxy-2-oxo-1,2-dihydroquinoline-3-carbonyl) glycine with co-povidone.

The XRPD pattern is as set forth in FIG. 4.

Particle size distribution: D₉₀=23.3 μm, D₅₀=11.2 μm, D₁₀=2.73 μm.

HPLC analysis of amorphous solid dispersions of compound of Formula I with co-povidone (1:3 w/w):
HPLC purity: 99.86% of compound of Formula I;
Content of compound of Formula A: 0.01%;
Content of compound of Formula B: Not detected;
Content of compound of Formula C: Not detected;
Content of compound of Formula D: 0.09%.

### Stability Data:

| Duration | |
|---|---|
| Initial | 15 days |

1. Storage Condition: 5° C. ± 3° C.

| | | |
|---|---|---|
| HPLC Purity | 99.86% | 99.85% |
| Polymorphic form | — | Unchanged |
| | | (Amorphous) |
| Content of Compound of Formula A | 0.01% | 0.03% |
| Content of Compound of Formula B | Not detected | Not detected |
| Content of Compound of Formula C | Not detected | Not detected |
| Content of Compound of Formula D | 0.09% | 0.09% |

2. Storage Condition: 25° C. ± 2° C. and 60% ± 5% Relative Humidity

| | | |
|---|---|---|
| HPLC Purity | | |
| Polymorphic form | — | Unchanged |
| | | (Amorphous) |
| Content of Compound of Formula A | 0.01% | 0.01% |
| Content of Compound of Formula B | Not detected | Not detected |
| Content of Compound of Formula C | Not detected | 0.01% |
| Content of Compound of Formula D | 0.09% | 0.08% |

### Example 5. Preparation of Amorphous Solid Dispersions of Compound of Formula I with Co-Povidone (1:1 w/w)

A mixture of (1-(cyclopropylmethoxy)-4-hydroxy-2-oxo-1,2-dihydroquinoline-3-carbonyl)glycine (2.5 g) and co-povidone (2.5 g) were dissolved in a mixture of methanol (50 mL) and dichloromethane (50 mL) at 25-35° C. The clear solution was fine filtered and used for spray drying. The solution was then spray dried to get an amorphous solid dispersion of (1-(cyclopropylmethoxy)-4-hydroxy-2-oxo-1,2-dihydroquinoline-3-carbonyl) glycine with co-povidone (1:1 w/w).

The XRPD pattern is as set forth in FIG. 5.

### Example 6. Preparation of Amorphous Solid Dispersions of Compound of Formula I with Co-Povidone (1:2 w/w)

A mixture of (1-(cyclopropylmethoxy)-4-hydroxy-2-oxo-1,2-dihydroquinoline-3-carbonyl)glycine (5 g) and co-povidone (10 g) were dissolved in a mixture of methanol (150 mL) and dichloromethane (150 mL) at 25-35° C. The clear solution was fine filtered and used for spray drying. The solution was then spray dried to get an amorphous solid dispersion of (1-(cyclopropylmethoxy)-4-hydroxy-2-oxo-1,2-dihydroquinoline-3-carbonyl) glycine with co-povidone (1:2 w/w).

The XRPD pattern is as set forth in FIG. 6.

### Example 7. Preparation of Amorphous Solid Dispersion of Compound of Formula I with HPMC-AS (1:3 w/w)

A mixture of (1-(cyclopropylmethoxy)-4-hydroxy-2-oxo-1,2-dihydroquinoline-3-carbonyl)glycine (2.5 g) and hydroxypropyl methylcellulose acetate succinate (HPMS-AS) (7.5 g) were dissolved in a mixture of methanol (120 mL) and dichloromethane (120 mL) at 25-35° C. The clear solution was fine filtered and used for spray drying. The solution was then spray dried to get an amorphous solid dispersion of (1-(cyclopropylmethoxy)-4-hydroxy-2-oxo-1,2-dihydroquinoline-3-carbonyl) glycine with HPMC-AS (1:3 w/w).

The XRPD pattern is as set forth in FIG. 7.

### Example 8. Preparation of Amorphous Solid Dispersion of Compound of Formula I with Kollidon® SR (1:1 w/w)

A mixture of (1-(cyclopropylmethoxy)-4-hydroxy-2-oxo-1,2-dihydroquinoline-3-carbonyl)glycine (2.5 g) and Kollidon® SR (2.5 g) were dissolved in a mixture of methanol (50 mL) and dichloromethane (50 mL) at 25-35° C. The clear solution was fine filtered and used for spray drying. The solution was then spray dried to get an amorphous solid dispersion of (1-(cyclopropylmethoxy)-4-hydroxy-2-oxo-1,2-dihydroquinoline-3-carbonyl) glycine with Kollidon® SR (1:1 w/w).

The XRPD pattern is as set forth in FIG. 8.

### Example 9. Preparation of Amorphous Solid Dispersion of Compound of Formula I with Kollidon® 90 (1:3 w/w)

The amorphous solid dispersions of compound of Formula I with Kollidon® 90 (1:3 w/w) was prepared by following the analogous process as described in example 8 above, except by taking Kollidon® 90 instead of Kollidon® SR in 1:3 w/w ratio.

The XRPD pattern is as set forth in FIG. 9.

### Example 10. Preparation of Amorphous Solid Dispersion of Compound of Formula I with Eudragit® RS-PO (1:3 w/w)

A mixture of (1-(cyclopropylmethoxy)-4-hydroxy-2-oxo-1,2-dihydroquinoline-3-carbonyl)glycine (2.5 g) and Eudragit® RS-PO (7.5 g) were dissolved in a mixture of methanol (120 mL) and dichloromethane (120 mL) at 25-35° C. The clear solution was fine filtered and used for spray drying. The solution was then spray dried to get an amorphous solid dispersion of (1-(cyclopropylmethoxy)-4-hydroxy-2-oxo-1,2-dihydroquinoline-3-carbonyl) glycine with Eudragit® RS-PO (1:3 w/w).

The XRPD pattern is as set forth in FIG. 10.

While the present invention has been described in terms of its specific embodiments, certain modifications and equivalents will be apparent to those skilled in the art and are intended to be included within the scope of the present invention.

1. An amorphous solid dispersion comprising a compound of Formula I, and 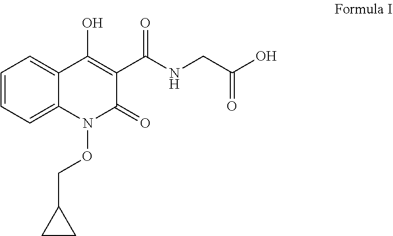 [SMILES: O=C(O)CNC(=O)c1c(O)c2ccccc2n(OCC2CC2)c1=O]
one or more pharmaceutically acceptable carriers.

2. The amorphous solid dispersion according to claim 1, wherein the one or more pharmaceutically acceptable carrier is selected from hydroxypropyl methylcellulose (HMPC), polyvinylpyrrolidone (PVP) or PVP based polymers (such as Kollidon® SR, Kollidon® 90), co-povidone, hydroxypropyl methylcellulose acetate succinate (HPMC-AS), hydroxypropyl cellulose, ethyl cellulose, carboxymethyl cellulose, polyethylene glycol, and copolymers based on methacrylic acid and methacrylic/acrylic ester or derivatives (such as Eudragit® RS-PO), or a mixture thereof.

3. The amorphous solid dispersion according to claim 1, wherein the ratio of the compound of Formula I to the pharmaceutically acceptable carrier is about 1:1 to about 1:10 w/w.

4. The amorphous solid dispersion according to claim 3, wherein the ratio of the compound of Formula I to the pharmaceutically acceptable carrier is about 1:1 to about 1:3 w/w.

5. The amorphous solid dispersion according to claim 1, wherein the amorphous solid dispersion is stable when stored at 2-8° C. or at 25±2° C./60±5% relative humidity for a period of 15 days or more, wherein the amorphous solid dispersion does not show any crystallinity after storage.

6. The amorphous solid dispersion according to claim 1, wherein the compound of Formula I is having a purity of about 98% or more, by area percentage of high-performance liquid chromatography (HPLC).

7. The amorphous solid dispersion according to claim 1, wherein the compound of Formula I is having a particle size of D₉₀ less than about 100 μm.

8. A process for the preparation of an amorphous solid dispersion according to claim 1,
the process comprising:
(a) preparing a solution of the compound of Formula I together with one or more pharmaceutically acceptable carriers in one or more solvents; and
(b) obtaining the amorphous solid dispersion by the removal of solvent.

9. The process according to claim 8, wherein the one or more pharmaceutically acceptable carrier comprises one or more of hydroxypropyl methylcellulose (HMPC), polyvinylpyrrolidone (PVP) or PVP based polymers (such as Kollidon® SR, Kollidon® 90), co-povidone, hydroxypropyl methylcellulose acetate succinate (HPMC-AS), hydroxypropyl cellulose, ethyl cellulose, carboxymethyl cellulose, polyethylene glycol, and copolymers based on methacrylic acid and methacrylic/acrylic ester or derivatives (such as Eudragit® RS-PO), or a mixture thereof.

10. The process according to claim 8, wherein the one or more solvent comprises one or more of chlorinated hydrocarbon solvents selected from dichloromethane, dichloroethane, and chlorobenzene; alcoholic solvents selected from methanol, ethanol, 2-propanol, 1-butanol, and t-butyl alcohol; or N,N-dimethylformamide; dimethyl sulfoxide; water; or mixtures thereof.

11. The process according to claim 10, wherein the solvent is a mixture of dichloromethane and methanol.

12. The process according to claim 8, wherein the removal of the solvent comprises one or more of distillation, distillation under vacuum, spray drying, agitated thin film drying (ATFD), freeze-drying (lyophilization), filtration, decantation, and centrifugation.

13. The process according to claim 12, wherein the removal of the solvent is by spray drying.

14. A pharmaceutical composition comprising an amorphous solid dispersion of a compound of Formula I, 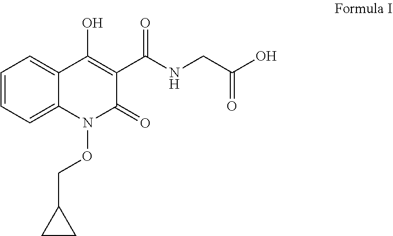 [SMILES: O=C(O)CNC(=O)c1c(O)c2ccccc2n(OCC2CC2)c1=O]
and one or more pharmaceutically acceptable excipients.

15. The pharmaceutical composition according to claim 14, wherein the compound of Formula I is having a purity of about 98% or more, by area percentage of high-performance liquid chromatography (HPLC).

16. A method for the treatment of anemia in a patient, comprising administering to a patient in need thereof a pharmaceutical composition according to claim 14.

17. A composition comprising an amorphous solid dispersion of compound of Formula I, 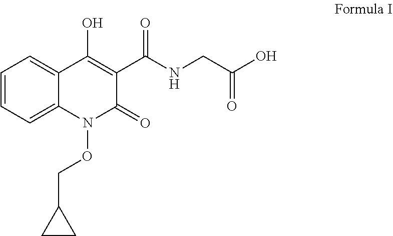 [SMILES: O=C(O)CNC(=O)c1c(O)c2ccccc2n(OCC2CC2)c1=O]
having a purity of about 99% or more, by area percentage of high-performance liquid chromatography (HPLC) together with one or more pharmaceutically acceptable carriers, and one or more of compounds of Formulae A, B, C, D or E present in an amount less than about 0.15% by area percentage of HPLC relative to the compound of Formula (I),

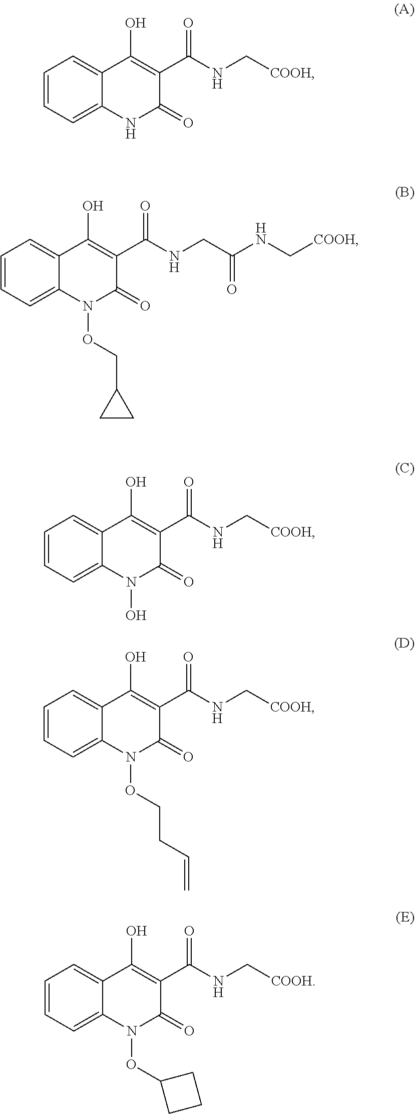

18. The composition according to claim 17, wherein the compound of Formula D is present in an amount less than about 0.15% by area percentage of HPLC relative to the compound of Formula (I).

19. The composition according to claim 17, wherein the compound of Formula E is present in an amount less than about 0.15% by area percentage of HPLC relative to the compound of Formula (I).
